(19) United States
(12) Patent Application Publication
(10) Pub. No.: US 2025/0134137 A1
(43) Pub. Date: May 1, 2025

(54) MAGNESIUM SUPPLEMENT FOR POULTRY

(71) Applicant: Timab Magnesium, Dinard (FR)

(72) Inventors: Marie-Sophie Schneegans, Dinard (FR); Julie Duclos, La Richardais (FR)

(73) Assignee: Timab Magnesium, Dinard (FR)

(21) Appl. No.: 19/004,979

(22) Filed: Dec. 30, 2024

Related U.S. Application Data
(63) Continuation of application No. 17/432,818, filed on Aug. 20, 2021.

(30) Foreign Application Priority Data
Feb. 21, 2019 (FR) 1901782

(51) Int. Cl. A23K 20/24; A23K 50/75

(52) U.S. Cl. CPC A23K 20/24; A23K 50/75

(57) ABSTRACT

The invention relates to a food supplement comprising at least one source of magnesium, one of its salts, one of its hydrates or one of its chelates, for the use thereof in the prevention and treatment of myopathies in poultry, or for the use thereof in the feeding of fast-growing poultry.

### TECHNICAL FIELD

The invention relates to a food additive for reducing the appearance of myopathies in poultry farms. This product contains at least one source of magnesium.

### PRIOR ART

Over the past decades, genetic selection of certain lines and the deployment of improved feeding programs has increased the speed of poultry growth and consequently shortened the production cycle. However, this improvement has been obtained at the cost of the quality of poultry meat.

The poultry industry is currently faced with a major challenge linked to emergent myopathies manifesting in the presence of white bands or striations parallel to the muscle fibers (white striping) but also in hardness of the meat (called “wooden breast” in the case of fillets). These two myopathies most frequently affect chicken fillets (pectoralis major) but may also be observed in the muscles of the thighs. Their incidence increases with the weight of the poultry, which is why they are more frequently observed in heavy poultry with high muscle mass (typically chickens whose weight at slaughter is greater on average than 2.4 kg). They pose no risk for human consumption but they do have a great impact on the composition of the meat (reduction in the protein content and increase in the lipid content) and on the technological properties of the meat, such as a reduction in the retention of water.

These myopathies lead to a high rate of downgrading per batch reared and even, for fillets exhibiting excessively great degrees of severity, in an inability to commercialize.

Various solutions involving the management of the feed have therefore been proposed and commercialized to reduce the incidence of these myopathies.

This is the case for various plant extracts having antioxidant activities, anti-inflammatory activities and/or activities stimulating blood circulation for better oxygenation of the muscle cells of the animals, and for a mixture comprising an antioxidant and a trace element (zinc, copper or manganese) chelated with methionine. The positive effect of the latter on the reduction in the number of chickens suffering from fillet hardness of grade 2 or 3 has been demonstrated taking an equivalent supplement of non-chelated trace elements as reference. These additives act on inflammation once the muscle cells have started to undergo oxidative stress.

There remains the need to be able to improve the quality of the poultry meat by taking action in advance of the anti-inflammatory reaction so as to improve the well-being of the animal. There also remains the need to propose additives for combating the myopathies without reducing the growth performance of the poultry.

The food supplement for poultry of the invention meets this need and comprises at least one source of magnesium.

The impact of magnesium supplementation has already been the subject of a study on poultry (J. Nelson, Feedstuffs, Vol. 87, No. 23, Jun. 22, 2015). It has been demonstrated in particular that magnesium increases the live weight of chickens while at the same time reducing the feed conversion ratio. A beneficial effect on bone strength has also been demonstrated. The supplementation of young hens and laying chickens with magnesium increases the live weight, bone strength and also the weight of eggs at the end of the egg-laying period.

Food additives comprising magnesium have also been described. For example, an additive containing magnesium lysinate in combination with other active agents such as plant extracts, chelates of zinc, of iron, of copper and of manganese, lysine, microorganisms and vitamin E enhances growth, improves the quality of the meat and of the eggs, and strengthens the immune system (CN 107518202A). Other additives comprising magnesium sulfate in combination with other minerals, proteins, acids, antibacterial agents, plant extracts and/or glucose make it possible to reduce the consumption index and the digestibility of the feed (CN 107136318A, CN 106376727A and CN 106798148A).

Now, the inventors have discovered in a highly surprising manner that magnesium makes it possible to reduce the appearance of myopathies in poultry farms without reducing the growth performance of the animals.

### DESCRIPTION OF THE INVENTION

A subject of the invention is therefore a food supplement comprising at least one source of magnesium, for the use thereof in the prevention and treatment of myopathies in farmed poultry.

According to one embodiment, the supplement is composed of at least one source of magnesium. Said supplement may be composed of magnesium oxide.

Magnesium supplementation of poultry advantageously makes it possible, in a preventative role, to reduce the incidence of myopathies in a farm. In particular, it makes it possible to reduce the number of animals suffering from myopathies without a negative impact on the growth performance of the farm. This result is all the more surprising since numerous authors have described the negative consequences of magnesium supplementation on the absorption of calcium and bone strength.

The prevention or treatment of myopathies within the meaning of the invention can correspond to the reduction in the incidence or the prevalence of myopathies observed in the farm compared to a non-supplemented farm. The prevalence can be calculated based on all of the myopathies observed or on the basis of a portion of them only. For example, the reduction in the prevalence of the myopathies may be reflected in the absence of poultry suffering from a severe myopathy.

The supplement may be used in the prevention of myopathies which risk harming farmed poultry, in particular broiler chickens. In other words, the supplement makes it possible to reduce the percentage of poultry affected by myopathies in a farm, in particular a farm of fast-growing poultry in which the prevalence of diseases is very high. Consequently, the invention is advantageously used in the feeding of fast-growing poultry. The poultry may be heavy chickens or turkeys. For the purposes of the invention, the term “heavy chicken” is understood to mean a chicken whose weight at slaughter is greater than or equal to 2.0 kg on average, or even greater than or equal to 2.4 kg on average.

Symptoms of myopathies may be the presence of white bands parallel to the muscle fibers in the fillets and the thighs of the poultry (white striping) or hardness of the fillets of the poultry (wooden breast). In a particular embodiment of the invention, the supplement makes it possible to reduce the number of animals affected by white striping, to reduce the number of animals affected by wooden breast, or to reduce both the number of animals affected by white striping and the number of animals affected by wooden breast.

For example, the incidence of white striping may be reduced by at least 30%, preferably by at least 40%, or indeed even by at least 45%. The incidence of wooden breast may be reduced by at least 15%, preferably by at least 20%, and, in certain embodiments, may be reduced by at least 45%.

In certain embodiments, the invention makes it possible to completely eliminate the occurrence of myopathies of degrees greater than or equal to 3 on a 4-point scale or a 5-point scale. Those skilled in the art will know how to evaluate the degree of severity of the myopathies wooden breast and white striping on the basis of their general knowledge, in particular by following the grading of the scales established by Kuttappan et al., 2012 Poultry Science 91:1240-1247, Kuttappan et al., 2016 Poultry Science 0:1-10 and Tijare et al., 2016 Poultry Science 0:1-7. These scales comprise several points, for example four points ranging from 0 (absence of myopathy) to 3 (severe myopathy).

The decrease in the incidence of the myopathies may be observed in particular relative to a control group for which the ration is not supplemented with a source of magnesium, said control group comprising at least 20%, at least 30%, or even at least 40% poultry suffering from myopathies at slaughter. The percentage reduction in myopathies will depend on the grade of severity of the myopathies and on the nature of the myopathies.

Depending on the case, all of the myopathies having a degree of severity ranging from 1 to 4, all of the myopathies having a degree of severity ranging from 2 to 4, all of the myopathies having a degree of severity of 3 or 4, or only the myopathies with a grade of 4 on a scale of 5 points can be used for the calculation of the reduction in myopathies. Depending on the case, all of the myopathies having a degree of severity ranging from 1 to 3, all of the myopathies having a degree of severity ranging from 2 to 3, or only the myopathies with a grade of 3 on a scale of 4 points can be used for the calculation of the reduction in myopathies. The percentage of myopathies of at least 20% may denote the percentage of white striping, the percentage of wooden breast, or the sum of the percentage of white striping and of the percentage of wooden breast.

In certain embodiments, the food supplement of the invention makes it possible to reduce the number of poultry having fillets affected by wooden breast (all degrees of severity combined) by at least 50%, and to reduce the number of poultry having fillets affected by white striping (all degrees of severity combined) by at least 50%, in particular in a population comprising 40% chickens suffering from myopathies.

In certain embodiments of the invention, the treatment makes it possible to eliminate the appearance of severe degrees of wooden breast, for example degrees greater than or equal to 3.

The reduction in the incidence of myopathies is advantageously obtained without a reduction in the growth performance of the animals and in particular without a reduction in the average weight at slaughter of the farmed population. The invention can reduce the number of fillets downgraded due to myopathy by at least 50% and/or increase the number of fillets completely free from myopathies by at least 10%. Lastly, the invention may improve the color of the fillets and/or the water holding capacity of the fillets.

The source(s) of magnesium may be chosen from magnesium salts, hydrates or chelates (Mg2+). They may be of natural or synthetic origin.

The following in particular are listed among the magnesium salts: magnesium oxide, magnesium carbonate, magnesium chloride, magnesium ascorbate, magnesium ascorbo-aspartate, magnesium L-aspartate, magnesium sulfate, magnesium stearate, magnesium silicate, magnesium pidolate, magnesium phosphate, magnesium acetate, magnesium orotate, magnesium malate, magnesium lactate, magnesium hydroxide, magnesium glycerophosphate, magnesium gluconate, magnesium citrate, magnesium nitrate, magnesium nitride, magnesium oleate, magnesium palmitate, magnesium sulfonate, magnesium carboxylate, magnesium borate, magnesium bromide, magnesium chromate, magnesium bisulfite, magnesium fluoride, magnesium iodate or iodide, magnesium permanganate, magnesium aluminate, magnesium salicylate, magnesium formate and magnesium tungstate.

Magnesium hydrates within the meaning of the invention denote all hydrated forms of any magnesium salt mentioned above.

Present among the magnesium chelates in particular are magnesium glycinate, magnesium bisglycinate, magnesium aspartate, magnesium alanate, magnesium glutamate, magnesium diglutamate, magnesium lysinate, magnesium methionate and magnesium threonate.

For example, the source of magnesium is magnesium oxide. The magnesium oxide may be obtained by mining, grinding and calcining a rock containing magnesium carbonate. The temperature of calcination of the rock can be between 550° C. and 1300° C. The particle size of the magnesium oxide obtained can be between 0.1 and 8 mm, and its purity can range from 70% to 99.9%.

The source of magnesium is preferably administered in an amount equivalent to a magnesium (Mg2+) dose ranging from 0.01% to 0.4% by mass, preferably ranging from 0.05% to 0.2% by mass, relative to the mass of solids in the feed ration given to the poultry.

The application additionally describes
a food supplement comprising at least one source of magnesium for the rearing of fast-growing poultry,
a method for rearing fast-growing poultry consisting in adding to the feed ration of said poultry in the farm an additive composed of at least one source of magnesium, and
the use of a food supplement composed of at least one source of magnesium for the feeding of fast-growing poultry.

Fast-growing poultry are in particular heavy chickens as defined above.

The invention is illustrated in more detail by the following examples.

### Example 1

The effect of magnesium oxide supplementation on the degree of incidence of myopathies in broiler chickens was studied.

### Conditions Under Which the Study was Carried Out:

For this, 270 chickens (males and females mixed) of the Ross 308 breed were divided randomly as follows: a control group and a group receiving 0.62% magnesium oxide. The animals received the same feed over the starting period. The treatment was applied during the growing and finishing phases, that is to say from day 15 to day 42 of the rearing. The primary feed was formulated to achieve or exceed the requirements recommended by the NRC (1994). Water and feed were dispensed freely. The temperature was set at 33-35° C. at the start of the study and was reduced by 3° C. per week to reach 24° C. as from the 21st day of life. 48 hours before slaughter, heat stress was applied to all treated animals (increase of the temperature to 30° C. for 8 h per day with an interval of 3-4 hours).

### Evaluations:

Growth performance was recorded on days 0, 14, 28 and 42. At the end of the study, 32 chickens were selected from each group to have homogeneous weights at slaughter (≈2.4 kg±0.2 kg). The fillets from these chickens were removed and immediately transferred for scoring of the wooden breast and white striping myopathies according to a 5-point grid. Meat quality parameters were recorded, such as the water retention (by the centrifugation method of Carvalho et al (2017)), the pH (AOAC method) and the color (carried out using a MINOLTA colorimeter and using the indicators redness, lightness and yellowness).

### Results:

Magnesium supplementation had no negative influence on the growth performance, health, or mortality. It made it possible to reduce the incidence of white striping by 43% (all degrees of severity combined) and the incidence of wooden breast by 50% (all degrees of severity combined) compared to the control group. Magnesium supplementation had a significant positive effect on meat color and water holding capacity compared to the control group. Fillets which had received the magnesium supplementation were redder (the redness value for the control group and the supplemented group was 3.81 and 4.06, respectively), less pale (the lightness value for the control group and the supplemented group was 51.76 and 53.89, respectively) and less yellow (the yellowness value for the control group and the supplemented group was 2.18 and 1.34, respectively). The water holding capacity (WHC) for the control group and the supplemented group was 88.67 and 94.37, respectively.

### Example 2

The effect of magnesium oxide supplementation on the meat quality of broiler chickens was studied.

### Conditions Under Which the Study was Carried Out:

For this, 200 chickens (males only) of the Ross 308 breed were divided randomly into two experiment treatment groups: a control group and a group treated with 0.3% magnesium oxide. The animals received the same feed over the starting period. The treatment was applied during the growing and finishing phases, that is to say from day 15 to day 43 of the rearing. The primary feed was formulated to achieve or exceed the requirements recommended by the genetics company. Water and feed were dispensed freely.

### Evaluation:

Growth performance was recorded on days 0, 12, 21, 28, 35 and 43. At the end of the study, 50 chickens per group were selected to have homogeneous weights at slaughter (≈3.15 kg±0.3 kg). The fillets from these chickens were removed and immediately transferred for scoring of the wooden breast and white striping myopathies according to a 3-point grid. Meat quality parameters were recorded, such as the water holding capacity (exudate method), the pH at 24 h and at 7 days post-mortem, and the color (Minolta CR400 chromameter using the indicators L*, a* and b*).

### Results:

Magnesium supplementation had no negative influence on the growth performance of the animals, the health, or the mortality. The treatment made it possible to reduce the incidence of white striping (all degrees of severity combined) by 52%. It also made it possible to reduce the incidence of wooden breast (all degrees of severity combined) by 21%. It should be noted that the treatment made it possible to eliminate the appearance of severe degrees of wooden breast. The treatment in particular made it possible to reduce the number of fillets downgraded due to myopathies by 62% and to increase the number of fillets completely free from myopathies by 11%.

The percentage of exudation for the control group and the supplemented group was 3.7% and 3.5%, respectively. The L* value for the control group and the supplemented group was 50.4 and 51.7, respectively. The b* value for the control group and the supplemented group was 0.0 and 0.8, respectively.

### Example 3

The effect of magnesium oxide supplementation on the fillet tenderness of broiler chickens was studied.

### Conditions Under Which the Study was Carried Out:

For this, 400 chickens (males only) of the Ross 308 breed were divided randomly into two experiment treatment groups: a control group and a group treated with 0.3% magnesium oxide. The animals received the same feed over the starting period. The treatment was applied during the growing and finishing phases, that is to say from day 15 to day 45 of the rearing. The primary feed was formulated to achieve or exceed the requirements recommended by the genetics company. Water and feed were dispensed freely.

### Evaluation:

Growth performance was recorded on days 0, 12, 21, 28, 35 and 45. At the end of the study, 40 chickens per group were selected to have homogeneous weights at slaughter (≈3.68 kg±0.1 kg). The fillets of these chickens were removed to measure fillet tenderness (carried out on the day following slaughter). The measurement was carried out by a physical method using a compression machine (3 repetitions per fillet) to measure two parameters: the compression force of the fillets and the hardness of the fillets (compression force as a function of the height of the fillet).

### Results:

Magnesium supplementation had no negative influence on the growth performance of the animals, the health, or the mortality. The treatment made it possible to improve the tenderness of the fillets. The compression force for the control group and the supplemented group was 9.93 N and 8.13 N, respectively. The hardness of the fillets for the control group and the supplemented group was 3.35 N/mm and 2.74 N/mm, respectively.

1-9. (canceled)

10. A method for reducing white striping in farmed poultry, the method comprising:
feeding poultry a feed composition comprising:
primary feed; and
supplemental magnesium;
wherein the percentage of poultry affected by white striping is reduced by at least 30% relative to poultry receiving the primary feed in the absence of the supplemental magnesium, and
wherein the method does not reduce growth performance of the poultry.

11. The method as claimed in claim 10, wherein the poultry are heavy chickens.

12. The method as claimed in claim 10, wherein the supplemental magnesium is in a form of one or more of magnesium salts, hydrates and/or chelates.

13. The method as claimed in claim 12, wherein the supplemental magnesium is in the form of magnesium oxide.

14. The method as claimed in claim 13, further comprising:
mining, grinding and calcining a rock containing magnesium carbonate, thereby obtaining the magnesium oxide.

15. The method as claimed in claim 14, wherein the magnesium oxide has a particle size between 0.1 and 8 mm, and a purity of the magnesium oxide ranges from 70% to 99.9%.

16. The method as claimed in claim 10, wherein the supplemental magnesium is administered in an amount equivalent to a magnesium dose ranging from 0.01% to 0.4% by mass, relative to a mass of solids in the feed composition given to the poultry.

17. The method of claim 10, wherein the poultry are turkeys.

18. The method of claim 10, wherein the supplemental magnesium is administered in an amount equivalent to a magnesium dose ranging from 0.05% to 0.4% by mass, relative to the mass of solids in the feed ration given to the poultry.

19. The method of claim 10, wherein the percentage of poultry affected by white striping is reduced by at least 40% relative to poultry receiving the primary feed in the absence of the supplemental magnesium.

20. The method of claim 10, wherein the percentage of poultry affected by white striping is reduced by at least 45% relative to poultry receiving the primary feed in the absence of the supplemental magnesium.

21. The method of claim 10, wherein the percentage of poultry affected by white striping is reduced by at least 50% relative to poultry receiving the primary feed in the absence of the supplemental magnesium.

22. The method of claim 10, wherein the growth performance is the average weight at slaughter of the farmed population.

23. The method of claim 10, wherein poultry is fast-growing poultry.

24. A method for reducing wooden breast in farmed poultry, the method comprising:
feeding poultry a feed composition comprising:
primary feed; and
supplemental magnesium.
wherein the percentage of poultry affected by wooden breast is reduced by at least 15% relative to poultry receiving the primary feed in the absence of the supplemental magnesium, and
wherein the method does not reduce growth performance of the poultry.

25. The method as claimed in claim 24, wherein the magnesium oxide has a particle size between 0.1 and 8 mm, and a purity of the magnesium oxide ranges from 70% to 99.9%.

26. The method as claimed in claim 24, wherein the supplemental magnesium is administered in an amount equivalent to a magnesium dose ranging from 0.01% to 0.4% by mass, relative to a mass of solids in the feed composition given to the poultry.

27. The method of claim 24, wherein the percentage of poultry affected by wooden breast by at least 45% relative to poultry receiving the primary feed in the absence of the supplemental magnesium.

28. The method of claim 24, wherein the percentage of poultry affected by wooden breast by at least 50% relative to poultry receiving the primary feed in the absence of the supplemental magnesium.

29. A food supplement comprising at least one source of magnesium, wherein the food supplement reduces white striping and wooden breast in farmed poultry without reducing growth performance.
